(19) United States
(12) Patent Application Publication
(10) Pub. No.: US 2026/0347470 A1
(43) Pub. Date: Nov. 5, 2026

(54) COMBINATION THERAPIES UTILIZING MICROBIALLY-EXPANDED GAMMA DELTA T CELLS AND T CELL ENGAGING BIOLOGICS

(71) Applicant: Providence Health & Services - Oregon, Portland, OR (US)

(72) Inventors: Keith Bahjat, Portland, OR (US); Marka Crittenden, Portland, OR (US)

(73) Assignee: Providence Health & Services - Oregon, Portland, OR (US)

(21) Appl. No.: 19/123,526

(22) PCT Filed: Oct. 26, 2023
(86) PCT No.: PCT/US2023/077946

§ 371 Date: Apr. 23, 2025

Related U.S. Application Data
(60) Provisional application No. 63/419,554, filed on Oct. 26, 2022.

(51) Int. Cl. C07K 16/28; A61K 31/661; A61K 35/74; A61K 40/33

(52) U.S. Cl. CPC C07K 16/2809; A61K 31/661; A61K 35/74; A61K 40/33; C07K 16/2887; A61K 2239/48; C07K 2317/31; C07K 2317/622

(57) ABSTRACT

Methods of treating a disease or disorder including administering to a subject a microbial conditioning regimen that activates, expands, and/or maintains a population of Vγ9Vδ2 T cells and administering to the subject a multi-specific T cell engager composition targeting an antigen expressed in the disease or disorder are provided. Methods of treating a disease or disorder including administering to a subject a population of ex vivo expanded Vγ9Vδ2 T cells or a population of engineered Vγ9Vδ2 T cells expressing a chimeric antigen receptor (CAR) targeting the disease or disorder and administering to the subject a microbial conditioning regimen that activates, expands, and/or maintains the population of ex vivo expanded Vγ9Vδ2T cells or the population of engineered Vγ9Vδ2 T cells are also provided.

### CROSS REFERENCE TO RELATED APPLICATIONS

This application claims the benefit of U.S. Provisional Application No. 63/419,554, filed Oct. 26, 2022, which is incorporated herein by reference in its entirety.

### FIELD

This disclosure relates to the field of immunotherapy, particularly methods of treating a disease or disorder including eliciting a microbe-specific immune response in combination with an agent allowing redirection of the elicited response to recognize and act on cells expressing an antigen associated with the disease or disorder

### BACKGROUND

Current immunotherapeutics that block negative regulators of the immune system (e.g., checkpoint blockade) are efficacious, but require the presence of pre-existing tumor-specific T cells capable of recognizing the subject's tumor. Bispecific T cell engagers (such as bispecific antibodies in which one portion binds to CD3 or a T cell receptor on the subject's T cells) can overcome lack of tumor specificity, but rely on the presence of T cells of sufficient number and fitness to mediate the therapeutic effect. CAR-T cells can be highly effective in specific cancer types, but require significant time and expense to manufacture the therapeutic. Thus, there remains a need to develop additional effective therapies for cancer, as well as infectious diseases.

### SUMMARY

Disclosed herein are methods that activate a microbe-specific immune response in a subject, combined with an agent allowing the elicited response to recognize and act on cells expressing an antigen associated with a disease or disorder. These methods can provide an autologous, disease-specific immune effector cell therapy, which in some examples, does not require the time or cost associated with ex vivo immune cell engineering.

Provided herein are methods of treating a subject with disease or disorder, the methods including administering to the subject a microbial conditioning regimen that activates, expands, and/or maintains a population of Vγ9Vδ2 TCR-expressing T cells, and administering to the subject a multi-specific T cell engager composition targeting an antigen expressed in the disease or disorder, thereby treating the disease or disorder. In some examples, the multi-specific T cell engager composition also targets one or more stimulatory receptors on the Vγ9Vδ2 TCR-expressing T cells. In some examples, the microbial conditioning regimen is administered to the subject prior to administering the multi-specific T cell engager composition (such as about 7 days prior to administering the multi-specific T cell engager composition).

In some examples, the multi-specific T cell engager composition includes a bi-specific T cell engager or a tri-specific T cell engager. In some examples, the multi-specific T cell engager includes a first portion including a binding domain that specifically binds to CD3 (for example, an anti-CD3 single chain variable fragment) and a second portion including a binding domain that specifically binds to an antigen expressed in the disease or disorder. In other examples, the multi-specific T cell engager includes a first portion including a binding domain that specifically binds to a TCRγ chain, a TCRδ chain, or both and a second portion including a binding domain that specifically binds to an antigen expressed in the disease or disorder. In some examples, the second portion of the multi-specific T cell engager includes an scFv that specifically binds to an antigen expressed in the disease or disorder or includes a portion of a TCR targeting an antigen that is expressed in the disease or disorder.

Also provided are methods of treating a subject with a disease or disorder, which methods include administering to the subject a population of ex vivo expanded Vγ9Vδ2 T cells or a population of engineered Vγ9Vδ2 T cells expressing a chimeric antigen receptor (CAR) targeting the disease or disorder, and administering to the subject a microbial conditioning regimen that activates, expands, and/or maintains the population of ex vivo expanded γδ T cells or the population of engineered γδ T cells. In some examples, the microbial conditioning regimen is administered to the subject concurrent with or after administering the population of ex vivo expanded γδ T cells or the population of Vγ9Vδ2 T cells expressing the CAR targeting the disease or disorder.

The disclosed methods include administering to a subject with a disease or disorder a microbial conditioning regimen. In some examples, the microbial conditioning regimen includes administering to the subject one or more doses of a composition comprising a bacterium that produces (E)-4-hydroxy-3-methyl-but-2-enyl pyrophosphate (HMBPP). In some examples, the bacterium that produces HMBPP is a live attenuated bacterium. In one example, the bacterium that produces HMBPP is Listeria monocytogenes. In some examples, the composition including the bacterium that produces HMBPP includes about 1×10⁵ to about 1×10⁹ colony forming units (CFUs) of the bacteria. The microbial conditioning regimen may be administered to the subject orally, intravenously, subcutaneously, intradermally, or intranasally.

In some examples, the subject with a disease or disorder is a subject with cancer or an infectious disease. In particular examples, the cancer is a solid tumor (such as lung cancer, prostate cancer, breast cancer, ovarian cancer, gastric cancer, pancreatic cancer, or melanoma). In other examples, the cancer is a hematological malignancy (such as B cell lymphoma, multiple myeloma, acute lymphoblastic leukemia, acute myeloid leukemia, or non-Hodgkin's lymphoma). In some examples, the subject with cancer is further administered one or more therapies selected from the group consisting of surgery, radiation treatment, chemotherapy, and immunotherapy.

The foregoing and other features of the disclosure will become more apparent from the following detailed description, which proceeds with reference to the accompanying figures.

### BRIEF DESCRIPTION OF THE DRAWINGS

FIG. 1 is a schematic diagram illustrating expansion of γδ T cells using a live microbial adjuvant. A bacterium producing HMB-PP, the predominant ligand for human Vγ9Vδ2 T cells, is taken up by phagocytic cells in vivo. The bacterial HMB-PP, together with the inflammatory response elicited by bacterial PAMPs, provides stimulatory signals to Vγ9Vδ2 T cells leading to activation and expansion of the Vγ9Vδ2 T cell population.

FIG. 2 illustrates the mevalonate (MVA) pathway (left) and non-mevalonate (MEP) pathway (right) for prenyl pyrophosphate biosynthesis. HMBPP, in the context of specific membrane associated molecules such as the butyrophilins, is recognized by Vγ9Vδ2 T cell receptors.

FIGS. 3A-3C illustrate examples of the disclosed methods for treatment of cancer. FIG. 3A illustrates direct killing of tumor cells expressing NKG2D ligands following in vivo expansion and activation of Vγ9Vδ2 T cells. FIG. 3B illustrates a combination of in vivo expansion and activation of Vγ9Vδ2 T cells with a multi-specific T cell engager targeting a tumor-associated antigen. FIG. 3C illustrates a combination of CAR-Vγ9Vδ2 T cells with a microbial conditioning regimen. In this scenario, the CAR-Vγ9Vδ2 T cells are expanded and maintained after delivery to the patient by treatment with an HMB-PP-producing bacterium.

FIGS. 4A-4C are a series of panels showing the absolute frequency of peripheral blood Vγ9Vδ2 T cells in nine subjects treated intravenously with the indicated doses of live-attenuated Listeria monocytogenes.

FIGS. 5A-5C show antibody guided cytotoxicity. FIG. 5A shows absolute number of Vγ9Vδ2 T cells per ml of blood in a patient treated with live-attenuated Listeria monocytogenes. FIG. 5B shows the frequency of Vγ9Vδ2 T cells with the total lymphocyte and CD3⁺ T cell populations determined by flow cytometry. FIG. 5C is a graph showing percent killing of SK-BR3 cells incubated with PBMCs obtained from a patient before (baseline) and 49 days after inoculation with Listeria monocytogenes in the presence of a bispecific anti-erbB2, anti-CD3 antibody, compared to incubation without the antibody for each sample.

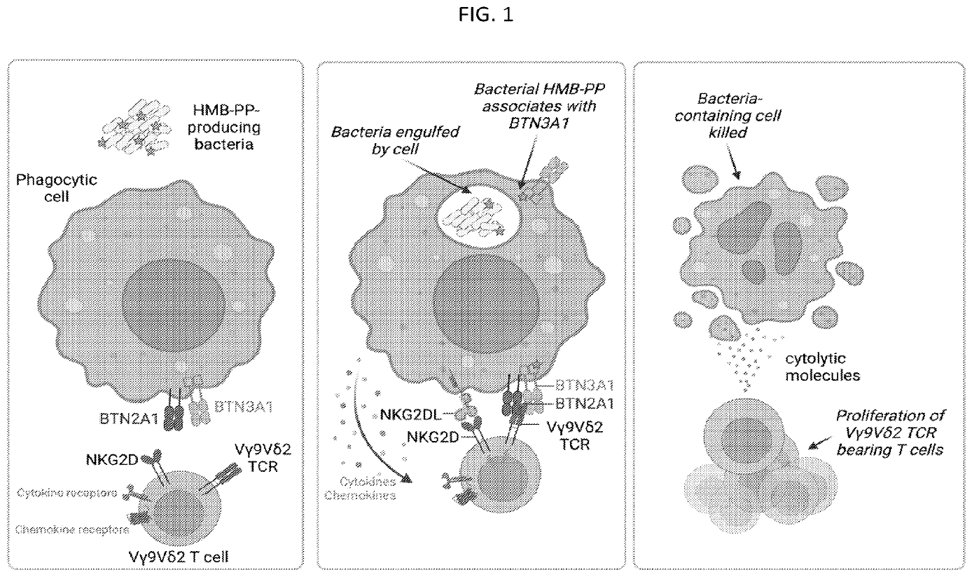

### DETAILED DESCRIPTION

Bacteria, such as Listeria monocytogenes (Lm), can be used to elicit an immune response in a variety of settings, for example, to deliver tumor-associated antigens, and to elicit activation, expansion, and maintenance of various T cell populations, including γδ T cells (such as Vγ9Vδ2 T cells), an innate-like population of T cells with potent cytotoxicity and cytokine production ability. It has been shown that treatment of patients with a live-attenuated microbe (such as Listeria monocytogenes) leads to activation, expansion, and maintenance of Vγ9Vδ2 T cells. These microbe-responsive invariant T cells can have cross-reactivity with cancerous cells. Human Vγ9Vδ2 T cells recognize invading pathogens through Pathogen Associated Molecular Patterns (PAMPs). One such PAMP is (E)-4-Hydroxy-3-methyl-but-2-enyl pyrophosphate (HMBPP), which together with butyrophilin family members BTN2A1 and BTN3A1, is recognized by the invariant Vγ9Vδ2 T cell receptor. Thus, HMBPP is a direct agonist of Vγ9Vδ2 T cells which allows for their rapid activation and expansion. There have been multiple attempts to engineer these bacteria as immunotherapeutic agents; however, the potency and robustness of such cross-reactivity appears insufficient to extend patient survival in a consistent manner. Provided herein is a combination of microbial conditioning to expand or activate Vγ9Vδ2 T cells in a subject, combined with multi-specific biologics to target the activated and expanded Vγ9Vδ2 T cells to diseased cells.

### I. TERMS

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Antibody: A polypeptide ligand comprising at least a light chain or heavy chain immunoglobulin variable region which recognizes and binds (such as specifically recognizes and specifically binds) an epitope of an antigen. Immunoglobulin molecules are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies known in the art, such as single-domain antibodies (e.g. V_{H} domain antibodies), Fab fragments, Fab′ fragments, F (ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies).

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs has been defined according to Kabat et al. (see, Kabat et al., Sequences of Proteins of Immunological Interest, U.S. Department of Health and Human Services, 1991) and ImMunoGeneTics database (IMGT) (see, Lefranc, Nucleic Acids Res 29:207-9, 2001; and imgt.cines.fr/IMGT_vquest/vquest?_livret=0&Option=humanIg). The Kabat database is maintained online (ncbi.nlm.nih.gov/igblast/). The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 (or H-CDR3) is located in the variable domain of the heavy chain of the antibody in which it is found, whereas a V_{L} CDR1 (or L-CDR1) is the CDR1 from the variable domain of the light chain of the antibody in which it is found. An antibody that binds PD-1, PD-L1, or PD-L2, for example, will have a specific V_{H} region and the V_{L} region sequence, and thus specific CDR sequences. Antibodies with different specificities (e.g., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “V_{H}” refer to the variable region of an immunoglobulin heavy chain, including that of an Fv, scFv, dsFv or Fab. References to “V_{L}” or “V_{L}” refer to the variable region of an immunoglobulin light chain, including that of an Fv, scFv, dsFv or Fab.

A “monoclonal antibody” is an antibody produced by a single clone of B-lymphocytes or by a cell into which the light and/or heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Humanized immunoglobulins can be constructed by means of genetic engineering.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and 2-dimensional nuclear magnetic resonance.

Autologous: Refers to tissues, cells or nucleic acids taken from an individual's own tissues. For example, in an autologous transfer or transplantation of T cells, the donor and recipient are the same person. Autologous (or “autogeneic” or “autogenous”) is related to self, or originating within an organism itself.

Chimeric antigen receptor (CAR): A chimeric molecule that includes an antigen-binding portion (such as a single domain antibody or scFv) and a signaling domain, such as a signaling domain from a T cell receptor (e.g. CD32). Typically, CARs include an antigen-binding portion, a transmembrane domain, and an intracellular domain. The intracellular domain typically includes a signaling domain having an immunoreceptor tyrosine-based activation motif (ITAM), such as CD3ζ or FcεRIγ. In some instances, the intracellular domain also includes the intracellular portion of at least one additional co-stimulatory domain, such as a co-stimulatory domain from CD28, 4-1BB (CD137), ICOS, OX40 (CD134), CD27, and/or DAP10.

Heterologous: Originating from a different genetic source. A nucleic acid molecule or protein that is heterologous to a cell originates from a genetic source other than the cell in which it is expressed, or is not normally expressed in the cell. In one specific, non-limiting example, a heterologous nucleic acid molecule encoding a protein (such as a CAR) is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Inhibiting or treating a disease: Inhibiting a disease, refers to inhibiting the full development of a disease or lessening the physiological effects of the disease process. In several examples, inhibiting or treating a disease refers to lessening symptoms of a tumor or an infectious disease. For example, cancer treatment can lessen a sign or symptom of the tumor, or infectious disease treatment can lessen a sign or symptom of the infectious disease. “Treatment” refers to a therapeutic intervention that ameliorates a sign or symptom of a disease or pathological condition related to the disease. The subject can be asymptomatic, so that the treatment prevents the development of a symptom. “Ameliorating” refers to the reduction in the number or severity of signs or symptoms of a disease, such as a tumor or an infectious disease.

Isolated: A biological component (such as a nucleic acid or protein, or cell) that has been substantially separated, produced apart from, or purified away from other biological components, for example other nucleic acids, proteins, and/or cells. Thus, isolated nucleic acids and proteins include nucleic acids and proteins purified by standard purification methods. The term also embraces nucleic acids and proteins prepared by recombinant expression in a host cell, as well as chemically synthesized nucleic acids. An isolated nucleic acid, protein, or cell can be at least 50%, at least 60%, at least 70%, at least 80%, at least 90%, at least 95%, at least 96%, at least 97%, at least 98%, or at least 99% pure.

Pharmaceutically acceptable carriers: The pharmaceutically acceptable carriers of use are known to those of ordinary skill in the art. Remington: The Science and Practice of Pharmacy, Adejare (Ed.), Academic Press, London, United Kingdom, 23rd Edition (2021) describes compositions and formulations suitable for pharmaceutical delivery of the disclosed agents. In general, the nature of the carrier will depend on the particular mode of administration being employed. For example, parenteral formulations usually include injectable fluids that include pharmaceutically and physiologically acceptable fluids such as water, physiological saline, balanced salt solutions, aqueous dextrose, glycerol or the like as a vehicle.

In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as in a selected quantity in a vial. Unit dosage forms can include one dosage or multiple dosages (for example, in a vial from which metered dosages of the agent can selectively be dispensed).

Purified: The term purified does not require absolute purity; rather, it is intended as a relative term. Thus, for example, a purified protein or cell preparation is one in which the protein or cell is more enriched than in its original environment. In one embodiment, a preparation is purified such that the protein or cells represent at least 50% of the total protein or cell content of the preparation.

Specific binding agent: An agent that binds substantially only to a defined target. The term “specifically binds” refers, with respect to an antigen, to the preferential association of an antibody or other ligand, in whole or part, with a cell or tissue bearing that antigen and not to cells or tissues lacking that antigen. It is, of course, recognized that a certain degree of non-specific interaction may occur between a molecule and a non-target cell or tissue. Nevertheless, specific binding may be distinguished as mediated through specific recognition of the antigen. Although selectively reactive antibodies bind antigen, they may do so with low affinity. Specific binding results in a much stronger association between the antibody (or other ligand) and cells bearing the antigen than between the antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a cell or tissue bearing the polypeptide as compared to a cell or tissue lacking the polypeptide. Specific binding to a protein under such conditions requires an antibody that is selected for its specificity for a particular protein. A variety of immunoassay formats are appropriate for selecting antibodies or other ligands specifically reactive with a particular protein. For example, solid-phase ELISA immunoassays can be routinely used to select monoclonal antibodies specifically immunoreactive with a protein.

Subject: Living multi-cellular vertebrate organisms, a category that includes human and non-human mammals, such as non-human primates, pigs, sheep, cows, dogs, cats, rodents, and the like. In an example, a subject is a human.

T cells: A white blood cell (lymphocyte) that is an important mediator of the immune response. T cells include, but are not limited to, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker.

γδ T cells are a subset of CD3⁺ T cells that express TCRs encoded by Vγ and Vδ gene segments. A particular subset of γδ T cells that is unique to humans and primates is Vγ9Vδ2 T cells. They constitute about 0.5-5% of the normal leukocyte population and expand dramatically in many acute infections. Vγ9Vδ2 T cells recognize a natural intermediate of the non-mevalonate isopentenyl pyrophosphate biosynthesis pathway expressed by many pathogens, (E)-4-hydroxy-3-methyl-but-2-enyl pyrophosphate (HMBPP).

In some examples, an “engineered T cell” is a T cell transduced with a heterologous nucleic acid (such as a nucleic acid or vector encoding a CAR) or expressing one or more heterologous proteins. The terms “engineered T cell,” “modified T cell,” and “transduced T cell” are used interchangeably in some examples herein.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. Thus, a metastatic cancer is a cancer at one or more sites in the body other than the site of origin of the original (primary) cancer from which the metastatic cancer is derived. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

### II. METHODS OF TREATMENT

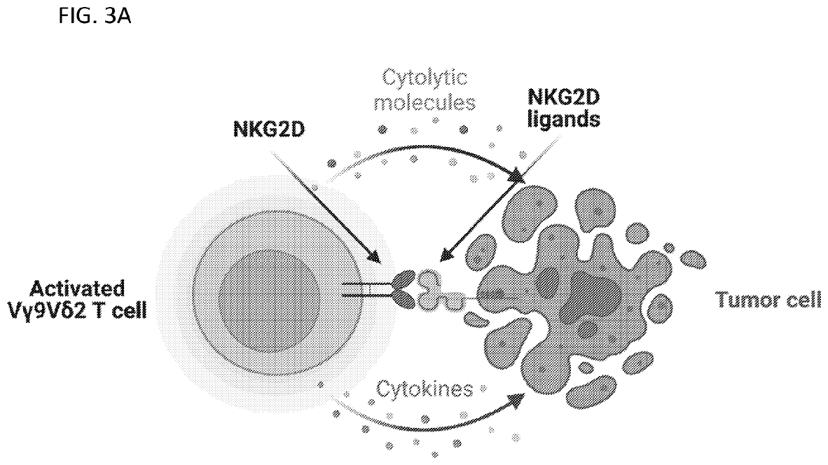

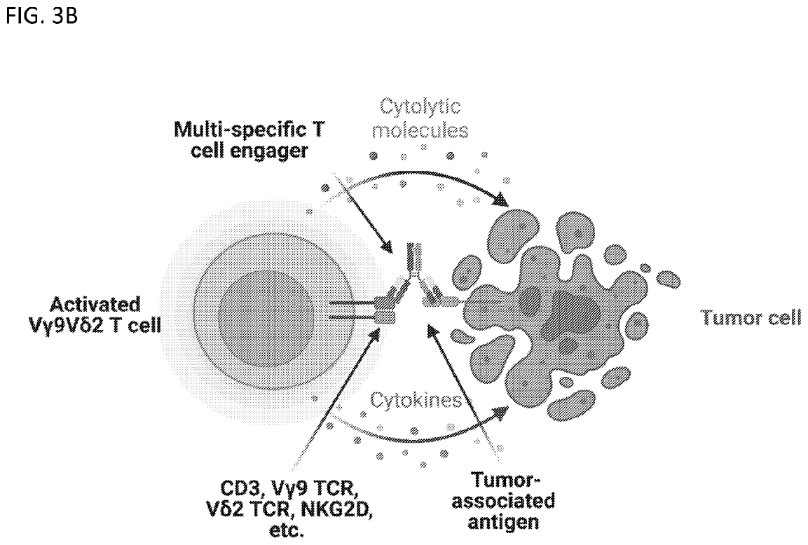

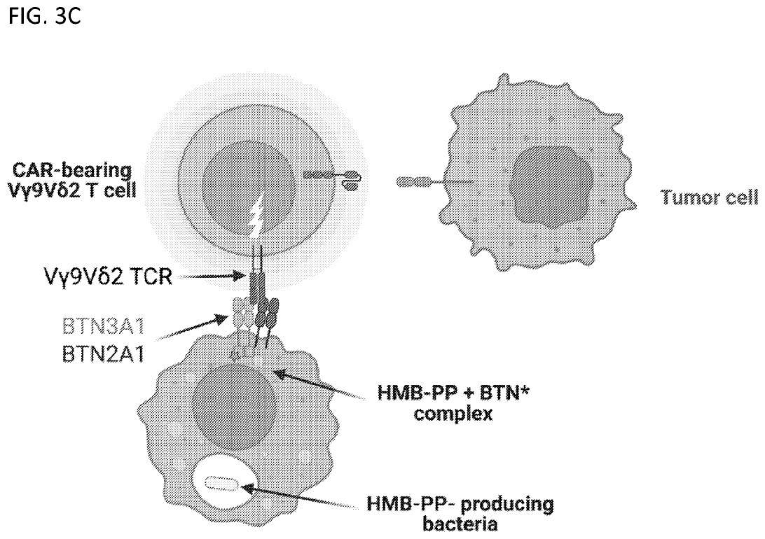

Provided herein are methods for treating a subject of interest, such as a subject with cancer or an infectious disease. The methods include combining a microbial conditioning regimen with a multi-specific (e.g., bi-specific, tri-specific, or tetra-specific) T cell engager (such as an antibody, nanobody, bi-specific T cell engager (BiTE)) that redirects the elicited microbe-specific immune response to recognize and act on diseased cells. In some examples, the subject is a human subject. Exemplary methods are illustrated in FIGS. 3A-3B.

In some examples, the methods include administering to the subject a microbial conditioning regimen that activates, expands, and/or maintains a population of Vγ9Vδ2 T cells and administering to the subject a multi-specific T cell engager composition targeting an antigen expressed in the disease or disorder. In some examples, additional therapeutics, such as additional cancer therapeutics, can be administered to the subject.

In several examples, the methods include administering the microbial conditioning regimen to the subject prior to administering the multi-specific T cell engager composition. In some examples, the microbial conditioning regimen is administered to the subject at least about 1 day prior to administering the multi-specific T cell engager composition, such as at least about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, or about 8 weeks prior to administering the multi-specific T cell engager composition. In other examples, the methods include administering the microbial conditioning regimen to the subject substantially simultaneously with administering the multi-specific T cell engager composition. In some examples, the microbial conditioning regimen is administered to the subject less than about 24 hours before administering the multi-specific T cell engager composition, for example, about 18 hours, about 16 hours, about 12 hours, about 8 hours, about 6 hours, about 4 hours, about 2 hours, about 1 hour or less before administering the multi-specific T cell engager composition.

In one example, the subject is administered one or more doses of the microbial conditioning regimen, such as 1, 2, 3, 4, or more doses. For example, multiple doses of the microbial conditioning regimen may produce increased or sustained γδ T cell response, compared to a single dose of the microbial conditioning regimen. In some examples, a second dose of the microbial conditioning regimen is administered to the subject after the first dose. In some examples, a second dose of the microbial conditioning regimen is administered about 7 days, 14 days, 21 days, or more after the first dose. Subsequent doses may be administered with the same timing as between the first and second dose, or with different timing (e.g., a longer or shorter interval). In some examples, the multi-specific T cell engager composition is administered after all doses of the microbial conditioning regimen. In other examples, the multi-specific T cell engager composition may be administered after at least one dose of the microbial conditioning regimen but prior to at least one subsequent dose of the microbial conditioning regimen.

In additional examples, the subject is administered one or more doses of the multi-specific T cell engager composition, such as 1, 2, 3, 4, or more doses. In some examples, the multi-specific T cell engager is administered by continuous infusion. In some examples, a second dose of the multi-specific T cell engager composition is administered about 1 week, 2 weeks, 3 weeks, 4 weeks, 5 weeks, 6 weeks, 7 weeks, 8 weeks, or more after the first dose. Subsequent doses may be administered with the same timing as between the first and second dose, or with different timing (e.g., a longer or shorter interval). In some examples, the multi-specific T cell engager composition is administered after all doses of the microbial conditioning regimen. In other examples, the multi-specific T cell engager composition may be administered after at least one dose of the microbial conditioning regimen but prior to at least one subsequent dose of the microbial conditioning regimen.

Multi-specific T cell engagers are molecules that are able to redirect T cells to target cancer (or other) cells, regardless of the T cells' antigen specificity. In particular examples, the disclosed methods utilize multi-specific T cell engagers to target γδ T cells to cells expressing a target antigen (such as a tumor antigen or an infectious disease antigen). In some examples, the multi-specific T cell engager composition is a bi-specific T cell engager or a tri-specific T cell engager. In examples, the multi-specific T cell engager includes a first portion that specifically binds to a T cell (such as a γδ T cell) and at least a second portion that specifically binds to an antigen expressed in the disease or disorder. In some examples, the multi-specific T cell engager further includes at least a third portion that specifically binds to an antigen expressed in the disease or disorder. The antigen may be the same or different from the antigen specifically bound by the second portion. Other molecules that redirect a T cell to recognize a target cell of interest may also be used in the methods provided herein. Exemplary additional types of multi-specific T cell engagers that may be used include dual affinity retargeting (DART) bi-specific antibodies, asymmetric IgG-based bi-specific T cell engagers, checkpoint-inhibitory T cell engagers (CiTE), simultaneous multiple interaction T cell engagers (SMITE), CAR-T cells engineered to co-express bi-specific T cell engagers (CART-BiTE), tri-specific killer engagers (TRIKE), and ImmTacs (see, e.g., Goebeler and Bargou, Nature Reviews Clinical Oncology 17:418-434, 2020).

In some examples, the first portion of the multi-specific T cell engager that specifically binds to a T cell is an antibody or portion thereof that binds to an antigen expressed by the T cell (such as a γδ T cell, for example, a Vγ9Vδ2 T cell). In certain examples, the antibody or portion thereof is a single chain variable fragment (scFv). In other examples, the first portion of the T cell engager is a nanobody, peptide, aptamer, anticalin, or butyrophilin complex that binds to the T cell or an antigen expressed by the T cell such as a γδ T cell, for example, a Vγ9Vδ2 T cell). In some examples, the first portion of the multi-specific T cell engager specifically binds to CD3, and in specific examples is an scFv that specifically binds to CD3. In other examples, the first portion of the multi-specific T cell engager specifically binds to NKG2D, DNAM-1, a γ TCR chain, or a 8 TCR chain. In some examples, the first portion of the multi-specific T cell engager is a protein or peptide that binds to NKG2D, such as an NKG2D ligand or portion thereof.

The second (or third, or more) portion of the multi-specific T cell engager binds specifically to an antigen that is expressed in the disease or disorder being treated. The antigen is referred to in some examples, as a target antigen. In some examples, the antigen is one expressed by a cancer. In other examples, the antigen in one that is expressed during an infection by an infectious agent. The antigen may be referred to as a tumor antigen, a tumor-associated antigen, or an infectious disease antigen. Exemplary target antigens are described in Section IV. The portion(s) of the multi-specific T cell engager that binds specifically to an antigen that is expressed in the disease or disorder in some examples is an antibody or portion thereof that binds specifically to an antigen that is expressed in the disease or disorder. In some examples, the antibody or portion thereof that binds specifically to an antigen that is expressed in the disease or disorder is an scFv. In other examples, the portion(s) of the multi-specific T cell engager that binds specifically to an antigen that is expressed in the disease or disorder is a portion of a TCR that targets the antigen expressed in the disease or disorder. In some examples, the multi-specific T cell engager includes an engineered (such as a soluble) TCR that specifically binds to a target antigen. In some examples, these molecules are referred to as immune mobilizing monoclonal T cell receptors against cancer (ImmTACs), and include a soluble, high affinity TCR where the two TCR chains are stabilized by a disulfide bond between the constant domains (see, e.g., Liddy et al., Nature Medicine 18:980-987, 2012). In additional examples, the portions of the multi-specific T cell engager that bind specifically to an antigen that is expressed in the disease or disorder include an antibody or portion thereof (such as an scFv) that binds to the antigen and a portion of a TCR that targets the antigen. In further examples, the multi-specific T cell engager includes a TCR mimic that binds to a specific MHC-peptide complex, similar to a TCR.

Also provided herein are methods of treating a subject with a disease or disorder that include administering to the subject a population of ex vivo expanded Vγ9Vδ2 T cells or a population of engineered Vγ9Vδ2 T cells expressing a chimeric antigen receptor (CAR) targeting the disease or disorder and administering to the subject a microbial conditioning regimen that activates, expands, and/or maintains the population of ex vivo expanded γδ T cells or the population of engineered γδ T cells.

An exemplary method is illustrated in FIG. 3C. In some examples, γδ T cells (such as Vγ9Vδ2 T cells) are transduced with a nucleic acid encoding a CAR that specifically binds to a target antigen (such as an antigen expressed by a cancer or an infectious disease) to produce a population of CAR-γδ T cells. The population of CAR-γδ T cells is administered to the subject. The population of CAR-γδ T cells may be expanded ex vivo prior to administration to the subject.

In some examples, the transduced or transfected cells are isolated T cells (such as a primary T cell or T cells obtained from a subject. In some examples, the T cells, are obtained from peripheral blood, umbilical cord blood, lymph node tissue, bone marrow, or tumor tissue. In some examples, the T cells are also enriched, purified, and/or expanded from a sample from a subject, for example before and/or after transduction with a CAR. In some examples, a γδ T cell-based cell therapy is generated from bone marrow, blood, tissue, or iPSCs. Cells originating from a complex cellular mixture are enriched prior to expansion of γδ T cells, and expansion may be carried out using zoledronic acid and IL-2, using TCR-binding agonist antibodies, or using an artificial antigen presenting cell expressing the γδ T cell stimulatory molecules. The T cells are autologous to the subject being treated or are allogeneic in nature.

In some examples, the subject is administered about 10⁴ to 10¹² CAR-γδ T cells (for example, about 10⁴-10⁸ cells, about 10⁶-10⁸ cells, or about 10⁶-10¹² cells). For example, the composition may be prepared such that about 10⁴ to 10¹⁰ modified cells/kg (such as about 10⁴, 10⁵, 10⁶, 10⁷, or 10⁸ cells/kg) are administered to a subject. In specific examples, the composition includes at least 10⁴, 10⁵, 10⁶, or 10⁷ CAR-γδ T cells. Multiple doses of the population of the CAR-Y& T cells can be administered to a subject. A skilled clinician can select an administration schedule based on the subject, the condition being treated, the previous treatment history, and other factors.

In several examples, the methods include administering the microbial conditioning regimen to the subject after administering the CAR-yd T cells to the subject. In some examples, the microbial conditioning regimen is administered to the subject at least about 1 day after administering the CAR-yd T cells, such as at least about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, or about 8 weeks after administering the CAR-γδ T cells. In other examples, the methods include administering the microbial conditioning regimen to the subject substantially simultaneously with administering the CAR-γδ T cells. In some examples, the microbial conditioning regimen is administered to the subject within about 24 hours of administering the CAR-γδ T cells, for example, within about 18 hours, about 16 hours, about 12 hours, about 8 hours, about 6 hours, about 4 hours, about 2 hours, about 1 hour or less of administering the CAR-γδ T cells.

In one example, the subject is administered one or more doses of the microbial conditioning regimen, such as 1, 2, 3, 4, or more doses. For example, multiple doses of the microbial conditioning regimen may produce increased or sustained CAR-γδ T cell response, compared to a single dose of the microbial conditioning regimen. In some examples, a second dose of the microbial conditioning regimen is administered about 7 days, 14 days, 21 days, or more after the first dose. Subsequent doses may be administered with the same timing as between the first and second dose, or with different timing (e.g., a longer or shorter interval).

### III. MICROBIAL CONDITIONING REGIMEN

The disclosed methods include a microbial conditioning regimen that activates, expands, and/or maintains a population of γδ T cells, such as Vγ9Vδ2 T cells. In some examples, the microbial conditioning regimen activates, expands, and/or maintains a population of Vγ9Vδ2 T cells in vivo. However, the methods may also include ex vivo expansion of γδ T cells (for example, Vγ9Vδ2 T cells) in certain examples.

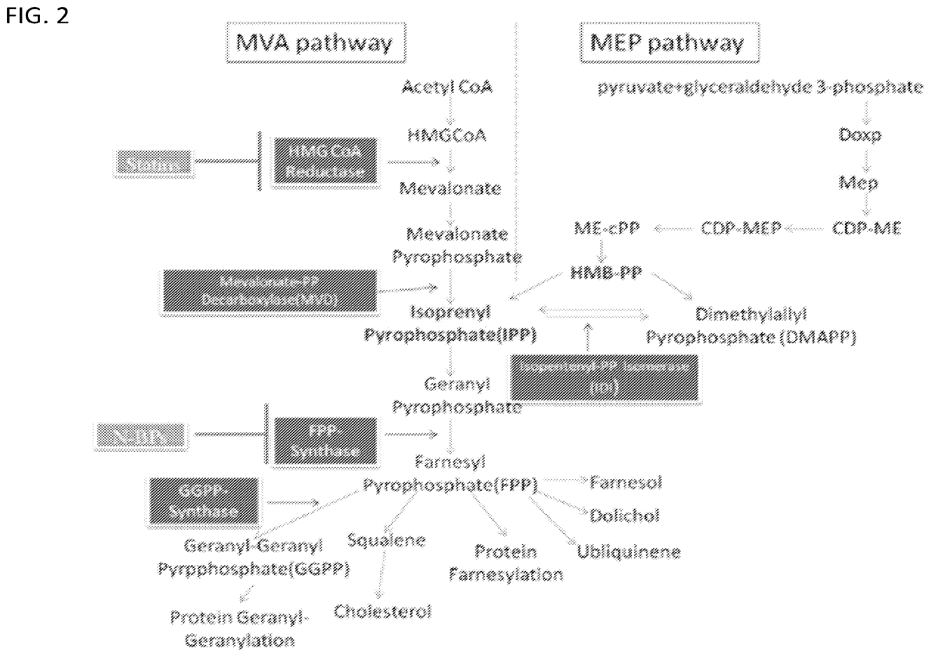

(E)-4-hydroxy-3-methyl-but-2-enyl pyrophosphate (HMBPP), which is an activator of Vγ9Vδ2 T cells, is an intermediate in the non-mevalonate (2C-methyl-D-erythritol 4-phosphate (MEP) pathway of isoprenoid biosynthesis (FIG. 2). Many bacteria utilize the MEP pathway for isoprenoid biosynthesis (either alone or in combination with the standard mevalonate pathway). Thus, the disclosed microbial conditioning regimens utilize one or more microbes that produce HMBPP in order to activate, expand, and/or maintain a population of Vγ9Vδ2 T cells. The microbes are live, and in some examples, are attenuated.

In some examples, the microbe is a gram-positive bacterium, such as Bacillus anthracis, Bacillus subtilis, Clostridium difficile, Clostridium botulinum, Clostridium perfringens, Listeria monocytogenes, or Nocardia terpenica. In other examples, the microbe is a gram-negative bacterium, such as Chlamydia trachomatis, Chlamydia pneumoniae, Salmonella enterica serovar Typhimurium, Escherichia coli (e.g., O157:H7 or O157: H6), Francisella tularensis, Pseudomonas aeruginosa, Vibrio cholerae, Klebsiella pneumoniae, Bordetella pertussis, Haemophilus influenzae, Helicobacter pylori, Shigella flexneri, Shigella dysenteriae, Neisseria gonorrhoeae, Neisseria meningitidis, Campylobacter jejuni, or Yersinia enterocolitica. In other examples, the microbe is Mycobacterium tuberculosis, Mycobacterium leprae, or Bacillus Calmette-Guerin (BCG). In some examples, the microbe is an intracellular pathogen, such as Listeria monocytogenes, Salmonella typhimurium, Francisella tularensis, Mycobacterium tuberculosis, or Chlamydia trachomatis. Exemplary microbes include Salmonella, Listeria, Shigella, Yersinia, and Escherichia. In one specific example, the microbe is Listeria monocytogenes, such as live attenuated Listeria monocytogenes. In some examples, the attenuated Listeria monocytogenes includes a deletion of the gene Acta, preventing polymerization of host cell actin and thus, preventing cell to cell spread, and a deletion of inlB, preventing direct infection of cells expressing the HGFR, such as hepatocytes (see, e.g., Brockstedt et al., Proc. Natl. Acad. Sci. USA 101:13832-13837, 2004). The microbe may also express one or more tumor antigens. In some examples, the microbe (such as Listeria monocytogenes) expresses NY-ESO-1 and EGFRvIII.

In other examples, the microbe is a microbe that does not endogenously produce HMBPP. The microbe may be engineered to produce HMBPP by introducing into the microbe one or more heterologous genes in the MEP pathway, leading to production of HMBPP. In some examples, the heterologous gene includes one or more (or all) of dxs, dxr, ispD, ispE, ispF, and ispG.

Methods of attenuating microbes (such as bacteria) are known in the art. Typically bacteria are attenuated using recombinant genetic techniques, for example to delete or mutate genes involved in virulence systems or to generate auxotrophic mutants (e.g., by deleting or mutating genes involved in biosynthesis of essential compounds such as aromatic amino acids, guanidine, or purine, for example, by allelic exchange).

In some examples, the microbial conditioning regimen includes administering to a subject a composition including an effective amount of a bacterium that produces HMBPP. The effective amount of the bacteria may be about 1 to about 1×10¹³ colony forming units (CFUs) of the bacteria (such as about 1 to about 100, about 10 to about 1×10³, about 100 to about 1×10⁴, about 1×10³ to about 1×10⁵, about 1×10⁴ to about 1×10⁶, about 1×10⁵ to about 1×10⁷, about 1×10⁶ to about 1×10⁸, about 1×10⁷ to about 1×10⁹, about 1×10⁸ to about 1×10¹⁰, about 1×10⁹ to about 1×10¹¹, about 1×10¹⁰ to about 1×10¹², or about 1×10¹¹ to about 1×10¹³ CFUs). For example, the effective amount may be about 1, about 5, about 10, about 50, about 100, about 500, about 1×10³, about 5×10³, about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, about 5×10¹⁰, about 1×10¹¹, about 5×10¹¹, about 1×10¹², about 5×10¹², or about 1×10¹³ CFUs.

The composition may include the effective amount of a bacterium that produces HMBPP and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, Adejare (Ed.), Academic Press, London, United Kingdom, 23rd Edition, 2021). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, Adejare (Ed.), Academic Press, London, United Kingdom, 23rd Edition (2021). The pharmaceutically acceptable carrier may be selected based on the route of administration of the composition.

In some examples, the composition is administered to the subject intravenously. In other examples, the composition is administered to the subject orally, subcutaneously, intradermally, or intranasally. An appropriate route of administration may be selected by a skilled clinician based on factors including the disease or disorder being treated, the condition of the subject, and other factors.

### IV. T CELL ENGAGER TARGETS

In some examples, the disclosed methods including administering to a subject a composition that can link a T cell to a target antigen, such as a target antigen expressed in a disease or disorder (for example, a tumor-associated antigen or an antigen expressed in an infectious disease). In some examples, the composition includes a multi-specific T cell engager or a multi-specific TCR binding composition that targets the disease or disorder being treated. In other examples, the composition may include a peptide, aptamer, or fAb receptor fusion protein (see, e.g., U.S. Pat. No. 11,098,093). In still further examples, the methods include administering to the subject a population of engineered γδ T cells expressing a chimeric antigen receptor (CAR) targeting the disease or disorder.

The target antigen is one expressed in the disease or disorder being treated. Exemplary target antigens and corresponding indications are shown in Table 1.

TABLE 1
Exemplary targets and indications

| Target | Indications |
|---|---|
| CD19 | Acute lymphoblastic leukemia (ALL), |
| | refractory or relapsed ALL, leukemia, |
| | lymphoma, B-cell lymphoma, T-cell |
| | lymphoma, Non-Hodgkin lymphoma, Diffuse |
| | large B-cell lymphoma (DLBCL), Primary |
| | mediastinal B-cell lymphoma, High grade B |
| | cell lymphoma, follicular lymphoma, DLBCL |
| | that results from follicular lymphoma, Chronic |
| | lymphocytic leukemia (CLL), Small |
| | lymphocytic leukemia (SLL), B-cell Non- |
| | Hodgkin lymphoma, B-cell Acute |
| | lymphoblastic leukemia, hematological cancer, |
| | acute myelogenous (myeloid) leukemia |
| | (AML), solid cancer, B-cell marginal zone |
| | lymphoma, Hodgkin's lymphoma, myeloma, |
| | multiple myeloma (MM), mantle cell |
| | lymphoma, Waldenstrom's |
| | hypergammaglobulinaemia, brain cancer, |
| | pancreatic cancer, gastrointestinal cancer, |
| | stomach cancer |
| CD22 | ALL, lymphoma, Non-Hodgkin lymphoma, |
| | autoimmune disease, DLBCL, CLL, B-cell |
| | lymphoma, leukemia |
| CD2 | Hematological cancer |
| CD4 | Leukemia, T-cell peripheral lymphoma, T-cell |
| | lymphoma, myeloma, MM |
| CD28 | Myeloma, MM |
| TNF receptor | Anaplastic large cell lymphoma, Hodgkin's |
| superfamily | lymphoma, T-cell peripheral lymphoma, T-cell |
| member 8 | lymphoma, non-Hodgkin's lymphoma |
| TNF receptor | Hematological cancer |
| superfamily | |
| member 9 | |
| TNF receptor | Solid cancer |
| superfamily | |
| member 10b | |
| TNF receptor | Hematological cancer, myeloma, solid cancer, |
| superfamily | MM |
| member 13B | |
| TNF receptor | Autoimmune disease, leukemia, lymphoma |
| superfamily | |
| member 13C | |
| B cell maturation | Myeloma, MM, myasthenia gravis, |
| factor | gastrointestinal cancer, stomach cancer, liver |
| (BCMA; | |
| TNF receptor | |
| superfamily | cancer, B-cell lymphoma, esophageal cancer, |
| member 17) | pancreatic cancer |
| CD171 | Neuroblastoma |
| Epidermal | Brain cancer, lung cancer, biliary cancer, non- |
| growth factor | small cell lung cancer (NSCLC), pancreatic |
| receptor (EGFR) | cancer, renal cancer, liver cancer, colorectal |
| | cancer |
| Epidermal growth | Glioblastoma |
| factor receptor | |
| variant III (EGFRvIII) | |
| Interleukin 3 | AML, Myelodysplastic syndrome, Blastic |
| receptor subunit | plasmacytoid dendritic cell neoplasm, |
| alpha (CD123; | Hodgkin's lymphoma, chronic myelogenous |
| IL3Ra) | (myeloid) leukemia (CML), hairy cell |
| | leukemia, mastocytosis, ALL, leukemia |
| Interleukin 7 receptor | Cancer |
| Interleukin 12 | Ovarian cancer, peritoneal cancer |
| receptor subunit | |
| beta 1 | |
| Interleukin-13 | Glioblastoma |
| receptor alpha | |
| (IL13Ra) | |
| Interleukin 13 | Brain cancer |
| receptor subunit | |
| alpha 2 | |
| Mesothelin | Ovarian cancer, cervical cancer, breast cancer, |
| | Fallopian tube cancer, pancreatic cancer, lung |
| | cancer, colorectal cancer, peritoneal |
| | carcinoma, solid cancer, mesothelioma, |
| | endometrial cancer |
| Mucin 16 (MUC-16) | Ovarian cancer, Peritoneal cancer |
| Mucin 1 (Muc1) | Sarcoma, breast cancer, cervical cancer, |
| | pancreatic cancer, lung cancer, liver cancer, |
| | glioma, colorectal cancer, gastric cancer, brain |
| | cancer, gastrointestinal cancer, stomach |
| | cancer, NSCLC, myeloma, MM, ovarian |
| | cancer, ovarian cancer, renal cancer, prostate |
| | cancer |
| Receptor tyrosine | Breast cancer, ovarian cancer, lung |
| kinase like | adenocarcinoma, lung cancer, lymphoblastic |
| orphan receptor | leukemia, CLL, ALL, NSCLC, mantle cell |
| 1 (ROR-1) | lymphoma |
| Prostate Stem | Pancreatic cancer, lung cancer, prostate cancer, |
| Cell Antigen | gastrointestinal cancer, stomach cancer, |
| (PSCA) | bladder cancer |
| CD33 | Myeloid leukemia, AML, hematological |
| | cancer, leukemia |
| Prostate specific | Prostate cancer, bladder cancer, cervical cancer |
| membrane | |
| antigen (PSMA) | |
| CD70 | B cell malignancies, breast cancer, ovarian |
| | cancer, pancreatic cancer, melanoma, renal cell |
| | cancer, B-cell lymphoma, T-cell lymphoma, |
| | renal cancer, non-Hodgkin's lymphoma |
| Human | Breast cancer, ovarian cancer, lung cancer, |
| epidermal growth | gastric cancer, colorectal cancer, pancreatic |
| factor receptor | cancer, glioblastoma, glioma |
| 2 (HER2) | |
| Carcinoembryonic | Breast cancer, lung cancer, colorectal cancer, |
| antigen | gastric cancer, pancreatic cancer, liver |
| (CEA) | metastases, liver cancer, peritoneal cancer, |
| | gastrointestinal cancer, stomach cancer, |
| | NSCLC |
| GTPase-activating | Solid tumors |
| protein | |
| (GAP) | |
| CD5 | T-cell ALL, T-cell non-Hodgkin lymphoma, |
| | T-cell peripheral lymphoma, leukemia, ALL, |
| | non-Hodgkin's lymphoma |
| CD38 | Myeloma, MM, AML |
| Ephrin type-A | Glioma |
| receptor 2 | |
| (EphA2) | |
| Fibroblast | Mesothelioma |
| activation protein | |
| alpha (FAP) | |
| Ganglioside G2 | Glioma, neuroblastoma, sarcoma, cervical |
| (GD2) | cancer, lung cancer, small cell lung cancer, |
| | melanoma, osteosarcoma |
| Epithelial | Breast cancer, prostate cancer, colon cancer, |
| cell adhesion | pancreatic cancer, gastric cancer, hepatic |
| molecule (EpCam) | carcinoma, esophageal carcinoma, lymphoma, |
| | leukemia, gastrointestinal cancer, stomach |
| | cancer, liver cancer, ovarian cancer |
| CD133 | AML, breast cancer, ovarian cancer, colorectal |
| | cancer, glioma, pancreatic cancer, liver cancer |
| Glypican 2 | Bladder cancer, small cell lung cancer, lung |
| | cancer, neuroblastoma |
| Glypican 3 | Lymphoma, leukemia, pancreatic cancer, |
| (GPC3) | colorectal cancer, lung cancer, liver cancer, |
| | brain cancer, breast cancer, gastrointestinal |
| | cancer, stomach cancer, NSCLC, squamous |
| | cell carcinoma |
| CD44 | AML, myeloma, MM, myeloma, breast cancer, |
| | colorectal cancer, lung cancer, leukemia |
| C-type lectin | AML, hematological cancer |
| domain family 12 | |
| member A (CLL1) | |
| C-type lectin domain | Solid cancer |
| containing 14A | |
| GDNF family | Thyroid cancer |
| receptor alpha 4 | |
| Membrane spanning 4- | Leukemia, Lymphoma, ALL, CLL, B-cell |
| domains A1 (CD20) | lymphoma, non-Hodgkin's lymphoma, |
| | DLBCL, melanoma, Waldenstrom's |
| | hypergammaglobulinaemia, brain cancer, |
| | pancreatic cancer |
| Programmed cell | Solid cancer, hematological cancer, esophageal |
| death 1 (PD-1) | cancer, DLBCL, non-Hodgkin's lymphoma, B- |
| | cell lymphoma |
| Erb-B2 receptor | Breast cancer, brain cancer, osteosarcoma, |
| tyrosine | sarcoma, ovarian cancer, colorectal cancer, |
| kinase 2 | gastrointestinal cancer, stomach cancer |
| Chondroitin sulfate | Brain cancer, breast cancer, neuroblastoma |
| proteoglycan 4 | |
| CD274 (PD-L1) | ALL, myeloma, MM, non-Hodgkin's |
| | lymphoma, pancreatic cancer, breast cancer, |
| | NSCLC, lung cancer |
| EPH receptor A2 | Solid cancer |
| EPH receptor A3 | Solid cancer |
| Folate hydrolase 1 | Prostate cancer |
| (PSMA) | |
| Folate receptor alpha | Ovarian cancer |
| Folate receptor beta | AML, solid cancer |
| Natural killer | Hematological cancer, solid cancer |
| cell cytotoxicity | |
| receptor 3 ligand 1 | |
| CD7 | AML, T-cell lymphoma, T-cell peripheral |
| | lymphoma, ALL, non-Hodgkin's lymphoma |
| KIT proto-oncogene, | Solid cancer |
| receptor | |
| tyrosine kinase | |
| Interleukin 6 | DLBCL |
| Interleukin 12A | Solid cancer |
| Interleukin 12B | Colorectal cancer |
| Interleukin 13 | Brain cancer |
| Interleukin 15 | CLL, B-cell lymphoma, hematological cancer |
| Fc fragment of | B-cell lymphoma |
| IgE receptor II | |
| Fc fragment of IgG | Hematological cancer, solid cancer |
| receptor IIIa | |
| Fms related | Hematological cancer, AML, solid cancer |
| tyrosine kinase 3 | |
| CD40 | B-cell lymphoma |
| Killer cell lectin | Bladder cancer, breast cancer, colorectal |
| like receptor | cancer, fallopian tube cancer, AML, NSCLC, |
| K1 | lung cancer, myeloma, ovarian cancer, |
| | pancreatic cancer, Myelodysplastic syndrome, |
| | hematological cancer |
| Cancer/testis | Solid cancer |
| antigen 1B | |
| MET proto- | Solid cancer |
| oncogene, receptor | |
| tyrosine kinase | |
| Thyroid stimulating | Thyroid cancer |
| hormone | |
| receptor (TSHR) | |
| T cell receptor | Anaplastic large cell cancer, non-Hodgkin's |
| beta constant 1 | lymphoma, T-cell lymphoma, T-cell |
| | peripheral lymphoma |
| T cell receptor | T-cell lymphoma |
| beta constant 2 | |
| AXL receptor | Renal cancer, bladder cancer, gastrointestinal |
| tyrosine kinase | cancer, stomach cancer, pancreatic cancer, soft |
| | tissue sarcoma |
| RAR related | Renal cancer, bladder cancer, gastrointestinal |
| orphan receptor A | cancer, stomach cancer, pancreatic cancer, soft |
| | tissue sarcoma |
| Alpha fetoprotein | Liver cancer |
| SLAM family | Myeloma, MM, B-cell lymphoma |
| member 7 | |
| (SLAMF7) | |
| Transforming | Prostate cancer |
| growth factor | |
| beta receptor 1 | |
| Transforming | Non-Hodgkin's lymphoma |
| growth factor | |
| beta receptor 2 | |
| Delta like | Small cell lung cancer, lung cancer |
| canonical Notch | |
| ligand 3 | |
| Claudin 3 | Colorectal cancer, ovarian cancer, pancreatic |
| | cancer, prostate cancer |
| Claudin 6 | Solid cancer |
| Claudin 18 | Gastrointestinal cancer, stomach cancer, |
| | pancreatic cancer, hematological cancer, |
| | ovarian cancer |
| Lewis Y (LeY) | Lung cancer, NSCLC |
| antigen | |
| L1 cell adhesion | Neuroblastoma, lung cancer, |
| molecule | pancreatic cancer, |
| (L1CAM) | renal cancer, brain cancer |
| Trophoblast | Solid cancer |
| glycoprotein | |
| Coagulation f | Hemophilia A |
| actor VIII | |
| Leukocyte | AL amyloidosis, LECT2 amyloidosis, |
| cell derived | transthyretin-related hereditary amyloidosis, |
| chemotaxin 2 | transthyretin-related wild-type amyloidosis, |
| (LECT2) | amyloidosis |
| Transthyretin | AL amyloidosis, LECT2 amyloidosis, |
| | transthyretin-related hereditary amyloidosis, |
| | transthyretin-related wild-type amyloidosis, |
| | amyloidosis |
| Muscle associated | Myasthenia gravis |
| receptor tyrosine | |
| kinase (MuSK) | |
| CD160 | CLL |
| Enhancer of Zeste | Solid cancer |
| 2 polycomb | |
| repressive | |
| complex 2 subunit | |
| (ENX) | |
| Sialophorin (CD43) | Leukemia, T-cell lymphoma |
| HIV-1 Env | HIV infection, AIDS |
| Complement | Gastrointestinal cancer, stomach cancer, |
| C3d receptor 2 | lymphoma, nasopharyngeal cancer, non- |
| (CR2) | Hodgkin's lymphoma |
| Kappa myeloma | Myeloma, MM |
| antigen | |
| (KMA) | |
| Lambda myeloma | Myeloma, MM |
| antigen | |
| (LMA) | |
| Kinase insert | Solid cancer |
| domain receptor | |
| (VEGFR2) | |
| Intercellular adhesion | Thyroid cancer |
| molecule 1 (ICAM-1) | |
| Aspartate | Hematological cancer, solid cancer |
| beta-hydroxylase | |
| (ASPH) | |
| Prominin 1 | Brain cancer |
| Cadherin 17 | Colorectal cancer, liver cancer, pancreatic |
| | cancer |
| Tumor associated | Colorectal cancer, liver cancer, pancreatic |
| calcium signal | |
| transducer 2 (Trop-2; | cancer |
| EGP-1) | |
| TRAF interacting | Solid cancer |
| protein | |
| (TRAIP) | |
| Tyrosinase (TYR) | Solid cancer, melanoma |
| Desmoglein 1 | Pemphigus |
| Desmoglein 3 | Pemphigus |
| Integrin beta 7 | Myeloma, MM |
| Adhesion G | AML, eosinophilic asthma, Churg-Strauss |
| protein coupled | syndrome, eosinophilic esophagitis |
| receptor E1 | |
| Gonadotropin | Ovarian cancer, prostate cancer, pancreatic |
| releasing | |
| hormone receptor | cancer |
| Tumor-associated | Colorectal cancer, ovarian cancer, prostate |
| glycoprotein | cancer |
| 72 (TAG-72) | |
| CD79a | B cell lymphoma, plasmacytoma, myeloma, |
| | Hodgkin's lymphoma |
| Six-transmembrane | Prostate cancer, bladder cancer, colon cancer, |
| epithelial | ovarian cancer, Ewing sarcoma |
| antigen of prostate | |
| (STEAP-1) | |
| Glycoprotein | Melanoma |
| 100 (gp100) | |

The multi-specific T cell engager is one that has at least a first portion that binds to a protein expressed by T cells (such as expressed by γδ T cells) and at least a second portion that binds to a target antigen expressed in the disease or disorder being treated. The multi-specific T cell engager may further include at least a third portion that binds to a second target antigen expressed in the disease or disorder. In some examples, the first portion is an antibody or portion thereof (such as an scFv) that binds to CD3. In some examples, the second portion is an antibody or portion thereof (such as an scFv) that binds to a target antigen expressed by a disease or disorder (including but not limited to those listed in Table 1). In a specific example, the multi-specific T cell engager includes at least a first portion that is an antibody or portion thereof (such as an scFv) that binds to CD3 and at least a second portion that is an antibody or portion thereof that binds to Her2 (also referred to as ErbB2). In one example, the second portion is trastuzumab. In other examples, the second portion is a TCR or portion thereof (such as a soluble TCR) that binds to a target antigen expressed by a disease or disorder (including but not limited to those listed in Table 1). Exemplary multi-specific T cell engagers and corresponding targets and indications are shown in Table 2.

TABLE 2
Exemplary multi-specific T cell engagers and indications.

| Name | Targets | Indications |
|---|---|---|
| AMG160 | CD3 × PMSA | Prostate cancer |
| AMG596 | CD3 × EGFRvIII | Glioblastoma |
| AMG757 | CD3 × DLL3 | Small-cell lung cancer |
| AMG199 | CD3 × MUC17 | Gastric cancer |
| AMG910 | CD3 × CLDN18.2 | Gastric cancer |
| AMV564 | CD3 × CD33 | Solid tumors |
| Blinatumomab | CD3 × CD19 | B cell precursor acute |
| | | lymphoblastic leukemia |
| Catumaxomab | EpCAM × CD3 | Gastric adenocarcinoma |
| Cibisatamab | CD3 × CEA | CEA-positive solid tumors |
| Elranatamab | BCMA × CD3 | Multiple myeloma |
| Epcoritamab | CD3 × CD20 | Diffuse large B cell lymphoma |
| | | (DLBCL) |
| ES414 | CD3 × PMSA | Prostate cancer |
| ERY974 | CD3 × GPC3 | Solid tumors |
| Flotetuzumab | CD3 × CD123 | Acute myeloid leukemia |
| GEN1044 | CD3 × 5T4 | Advanced or metastatic solid |
| | | tumors |
| Glofitamab | CD3 × CD20 | diffuse large B-cell lymphoma |
| | | (DLBCL) |
| HPN217 | CD3 × ALB × | Multiple myeloma |
| | BCMA | |
| HPN328 | CD3 × ALB × | Small cell lung cancer |
| | DLL3 | |
| HPN536 | CD3 × ALB × | late-stage ovarian, pancreatic, |
| | mesothelin | and mesothelioma cancers |
| HPN601 | CD3 × ALB × | Prostate, NSCLC, breast, colon, |
| | EpCAM | endometrial, thyroid, SCLC, |
| | | gastric, ovarian, esophageal, |
| | | neuroendocrine, and gallbladder |
| Hu3F8-bsAB | CD3 × GD2 | Neuroblastoma, osteosarcoma, |
| | | GD2-postive solid tumors |
| ISB 1302 | CD3 × HER2 | Her2-positive breast cancer |
| M802 | CD3 × HER2 | HER2-positive solid tumors |
| M701 | CD3 × EpCAM | Malignant ascites |
| MGD007 | CD3 × gpA33 | Colorectal carcinoma |
| Mosunetuzumab | CD3 × CD20 | Indolent and aggressive non- |
| | | Hodgkin's lymphoma; B cell |
| | | lymphoma |
| Odronextamab | CD3 × CD20 | B cell non-Hodgkin's lymphoma |
| Pasotuxizumab | CD3 × PSMA | Prostate cancer |
| REGN5458 | CD3 × BCMA | Hematological malignancies; |
| | | multiple myeloma |
| Talquetamab | CD3 × GPRC | Hematological malignancies; |
| | | Multiple myeloma |
| Tarlatamab | CD3 × DLL3 | Solid tumors |
| Teclistamab | CD3 × BCMA | Multiple myeloma |
| TNB-383B | CD3 × BCMA | Hematological malignancies; |
| | | relapsed/refractory multiple |
| | | myeloma |
| Tebentafusp | CD3 × HLA- | Melanoma |
| | A*02:01 | |
| | TCR domain | |
| | targeting gp100 | |
| 2A9-MICA | MICA × BCMA | Multiple myeloma |
| NKAB-ErbB2 | NKG2D × ErbB2 | Breast cancer |
| CS1-NKG2D | CS1 × NKG2D | Multiple myeloma |
| ULBP2-BB4 | NKGD × CD138 | Multiple myeloma |
| ULBP2-CEA | NKGD × CEA | Solid tumors, colon carcinoma |
| NKG2D-HER2 | NKG2D × HER2 | Breast cancer |

### V. DISEASES OR DISORDERS

In some examples, the subject being treated has a solid tumor. Examples of solid tumors include sarcomas (such as fibrosarcoma, myxosarcoma, liposarcoma, chondrosarcoma, osteosarcoma, soft tissue sarcoma, and other sarcomas), synovioma, mesothelioma, Ewing sarcoma, leiomyosarcoma, rhabdomyosarcoma, colon cancer, colorectal cancer, peritoneal cancer, esophageal cancer (such as esophageal squamous cell carcinoma), pancreatic cancer, breast cancer (including basal breast carcinoma, ductal carcinoma and lobular breast carcinoma), endometrial cancer, lung cancer (such as non-small cell lung cancer or small-cell lung cancer), ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioblastoma, astrocytoma, medulloblastoma, diffuse intrinsic pontine glioma, craniopharyrgioma, ependymoma, pinealoma, hemangioblastoma, acoustic neuroma, oligodendroglioma, meningioma, neuroblastoma and retinoblastoma). Solid tumors also include tumor recurrences and/or metastases (for example, metastases to the lung, liver, brain, or bone). In some particular examples, the subject has breast cancer, non-small cell lung cancer or prostate cancer.

In other examples, the subject has a hematological malignancy. Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia. In one particular example, the subject has B cell lymphoma.

In some examples, the subject with cancer is also treated with surgery, radiation therapy, chemotherapeutic agents, additional immunotherapies, or any combination thereof. A skilled clinician can select an appropriate combination of additional treatments with the methods provided herein, based on the type of cancer being treated.

In other examples, the subject has an infectious disease, such as a disease or disorder caused by a bacterial agent, viral agent, or parasitic agent. Exemplary infectious diseases include arboviral infections, botulism, brucellosis, candidiasis, campylobacteriosis, chickenpox, chlamydia, cholera, coronavirus infections, Staphylococcus infections, coxsackie virus infections, Creutzfeldt-Jakob disease, cryptosporidiosis, Cyclospora infection, cytomegalovirus infections, Epstein-Barr virus infection, dengue fever, diphtheria, ear infections, encephalitis, influenza virus infections, parainfluenza virus infections giardiasis, gonorrhea, Haemophilus influenzae infections, hantavirus infections, viral hepatitis, herpes simplex virus infections, HIV/AIDS, Helicobacter infection, human papillomavirus (HPV) infections, infectious mononucleosis, legionellosis, leprosy, leptospirosis, listeriosis, Lyme disease, lymphocytic choriomeningitis, malaria, measles, Marburg hemorrhagic fever, meningitis, monkeypox, mumps, mycobacteria infection, Mycoplasma infection, Norwalk virus infection, pertussis, pinworm infection, pneumococcal disease, Streptococcus pneumonia infection, Mycoplasma pneumoniae infection, Moraxella catarrhalis infection, Pseudomonas aeruginosa infection, rotavirus infection, psittacosis, rabies, respiratory syncytial virus infection (RSV), ringworm, rocky mountain spotted fever, rubella, Salmonellosis, SARS (such as SARS-COV or SARS-COV-2), scabies, sexually transmitted diseases, shigellosis, shingles, sporotrichosis, streptococcal infections, syphilis, tetanus, trichinosis, tuberculosis, tularemia, typhoid fever, viral meningitis, bacterial meningitis, west Nile virus infection, yellow fever, adenovirus-mediated infections and diseases, retrovirus-mediated infectious diseases and yersiniosis zoonoses.

In some examples, the subject with an infectious disease also treated with one or more additional therapeutics, such as antibiotics, antivirals, anti-parasitics, or other therapeutics. A skilled clinician can select an appropriate combination of additional treatments with the methods provided herein, based on the type of infectious disease being treated.

### VI. OVERVIEW OF SEVERAL ASPECTS

Aspect 1. A method of treating a subject with disease or disorder, comprising:
administering to the subject a microbial conditioning regimen that activates, expands, and/or maintains a population of Vγ9Vδ2 T cells; and
administering to the subject a multi-specific T cell engager composition targeting an antigen expressed in the disease or disorder and one or more stimulatory receptors expressed by the Vγ9Vδ2 T cells, thereby treating the disease or disorder.

Aspect 2. The method of aspect 1, wherein the microbial conditioning regimen is administered to the subject prior to administering the multi-specific T cell engager composition.

Aspect 3. The method of aspect 1 or aspect 2, wherein the microbial conditioning regimen is administered to the subject about 7 days prior to administering the multi-specific T cell engager composition.

Aspect 4. The method of any one of aspects 1 to 3, wherein the multi-specific T cell engager composition comprises a bi-specific T cell engager or a tri-specific T cell engager.

Aspect 5. The method of any one of aspects 1 to 4, wherein the multi-specific T cell engager comprises:
a first portion comprising a binding domain that specifically binds to CD3; and
a second portion comprising a binding domain that specifically binds to an antigen expressed in the disease or disorder.

Aspect 6. The method of aspect 5, wherein the first portion of the multi-specific T cell engager comprises an anti-CD3 single chain variable fragment (scFv).

Aspect 7. The method of aspect 5 or aspect 6, wherein the second portion of the multi-specific T cell engager comprises an anti-Her2 (ErbB2) antibody or fragment thereof.

Aspect 8. The method of any one of aspects 1 to 4, wherein the multi-specific T cell engager comprises:
a first portion comprising a binding domain that specifically binds to a TCRγ chain, a TCRδ chain, or both; and
a second portion comprising a binding domain that specifically binds to an antigen expressed in the disease or disorder.

Aspect 9. The method of any one of aspects 1 to 8, wherein the second portion of the multi-specific T cell engager comprises a single chain variable fragment (scFv) that specifically binds to the antigen expressed in the disease or disorder or comprises a portion of a TCR targeting an antigen that is expressed in the disease or disorder.

Aspect 10. A method of treating a subject with a disease or disorder, comprising:
administering to the subject a population of ex vivo expanded Vγ9Vδ2 T cells or a population of engineered Vγ9Vδ2 T cells expressing a chimeric antigen receptor (CAR) targeting the disease or disorder; and
administering to the subject a microbial conditioning regimen that activates, expands, and/or maintains the population of ex vivo expanded Vγ9Vδ2 T cells or the population of engineered Vγ9Vδ2 T cells.

Aspect 11. The method of aspect 10, wherein the microbial conditioning regimen is administered to the subject concurrent with or after administering the population of Vγ9Vδ2 T cells expressing the CAR targeting the disease or disorder.

Aspect 12. The method of any one of aspects 1 to 11, wherein the microbial conditioning regimen comprises administering to the subject one or more doses of a composition comprising a bacterium that produces (E)-4-hydroxy-3-methyl-but-2-enyl pyrophosphate (HMBPP).

Aspect 13. The method of aspect 12, wherein the bacterium that produces HMBPP is a live attenuated bacterium.

Aspect 14. The method of aspect 12 or aspect 13, wherein the bacterium that produces HMBPP is Listeria monocytogenes.

Aspect 15. The method of aspects 12 to 14, wherein the composition comprising the bacterium that produces HMBPP comprises about 1 to about 1×10¹³ colony forming units (CFU) of the bacteria.

Aspect 16. The method of any one of aspects 1 to 15, wherein the microbial conditioning regimen is administered orally, intravenously, subcutaneously, intradermally, or intranasally.

Aspect 17. The method of any one of aspects 1 to 16, wherein the disease or disorder is cancer or an infectious disease.

Aspect 18. The method of aspect 17, wherein the cancer is a solid tumor or a hematological malignancy.

Aspect 19. The method of aspect 18, wherein the cancer is a solid tumor comprising lung cancer, prostate cancer, breast cancer, ovarian cancer, gastric cancer, pancreatic cancer, or melanoma.

Aspect 20. The method of aspect 18, wherein the cancer is a hematological malignancy comprising B cell lymphoma, multiple myeloma, acute lymphoblastic leukemia, acute myeloid leukemia, or non-Hodgkin's lymphoma.

Aspect 21. The method of any one of aspects 17 to 20, wherein the subject has cancer and further comprising administering to the subject one or more therapies selected from the group consisting of surgery, radiation treatment, chemotherapy, and immunotherapy.

### EXAMPLES

The following examples are provided to illustrate certain features of the disclosure. These examples should not be construed to limit the disclosure to the particular features described.

### Example 1

### Expansion and Persistence of Vγ9Vδ2 T Cells In Vivo

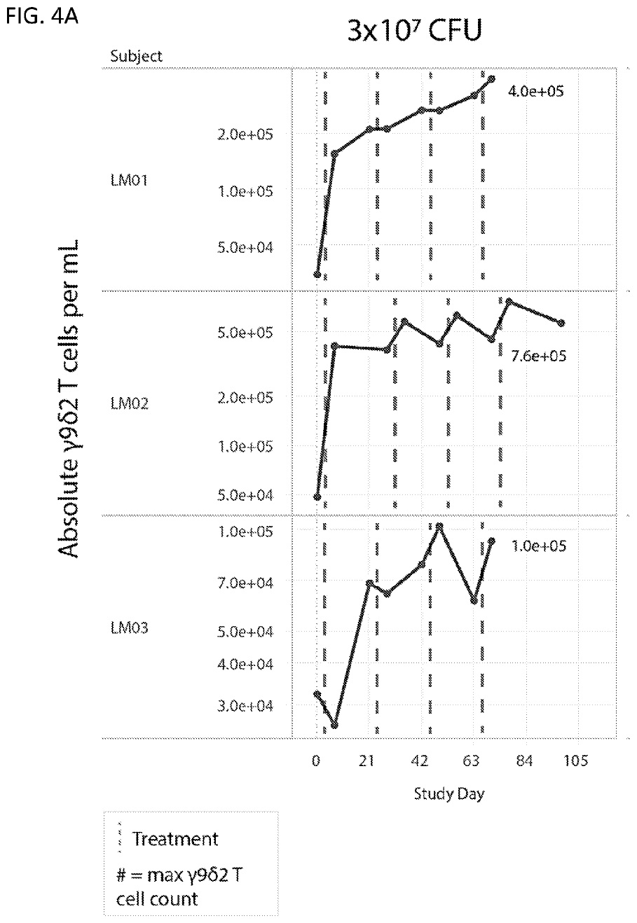

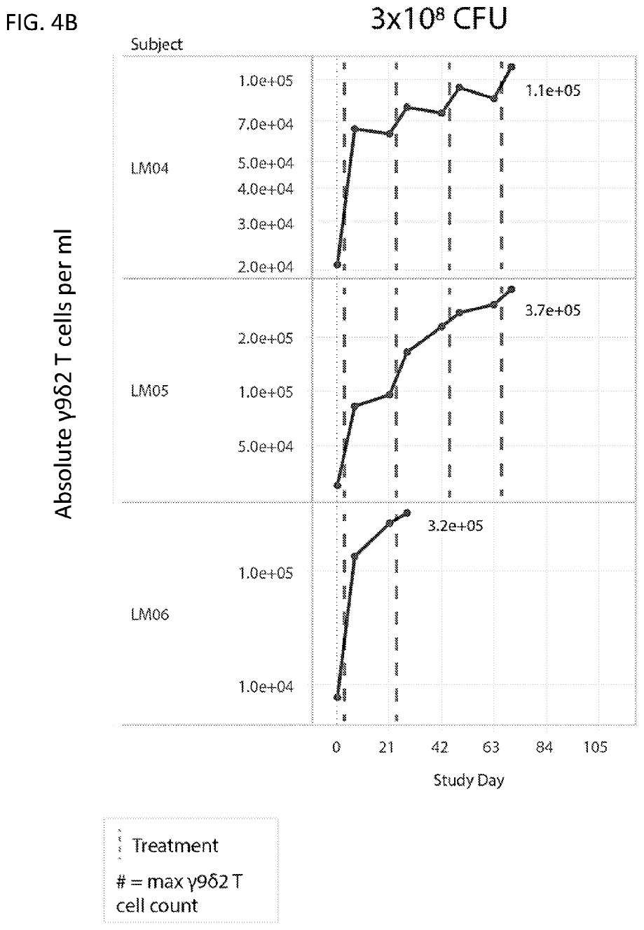

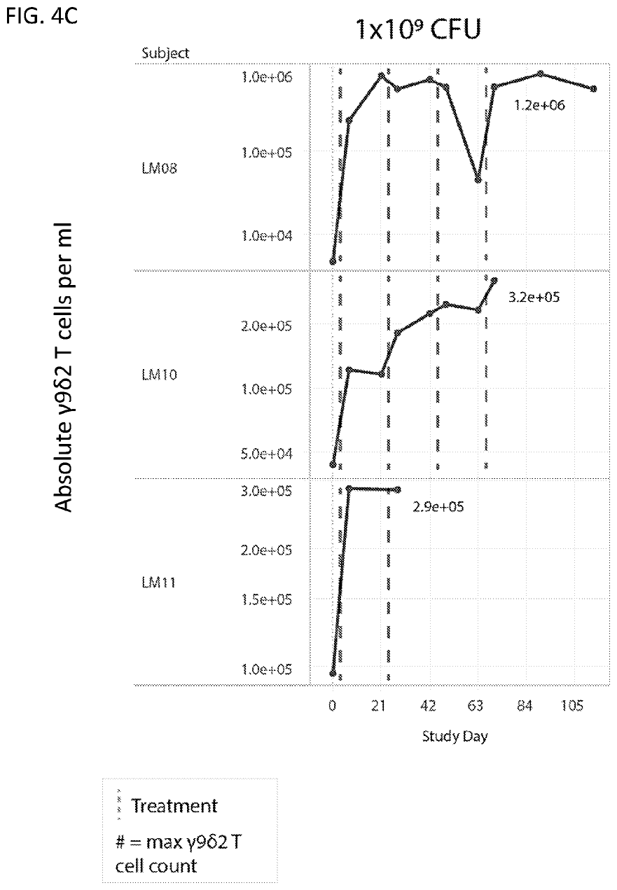

In a Phase I study of safety and immunogenicity, subjects with treated and recurrent Grade III/IV astrocytoma were administered ADU-623 on days 0, 21, 42, and 63. ADU-623 is a live attenuated Listeria monocytogenes derived from the 10403S strain. Deletions of actA and inlB prevent cell to cell spread and hepatocyte infection. The cells also expressed two tumor-associated antigens, EGFRvIII and NY-ESO1. Peripheral blood was collected prior to, 24 hours after, and 7 days after each dose. Activation, expansion, and maintenance of γ9δ2 T cells was observed, with expansion independent of dose (FIG. 4).

### Example 2

### Evaluation of Guided Cytotoxicity by γδ T Cells

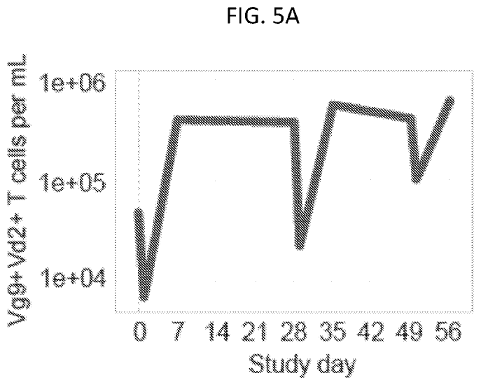

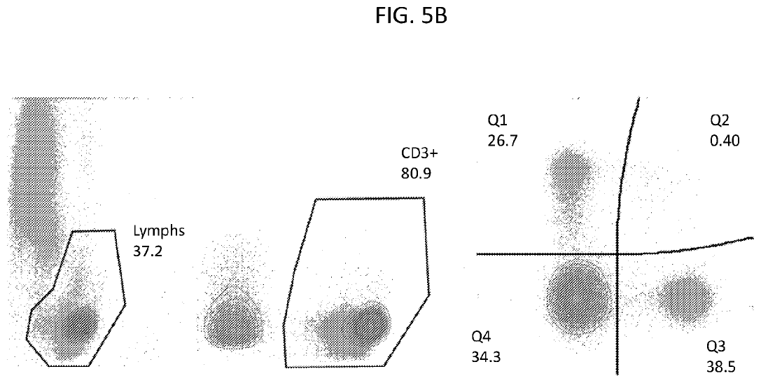

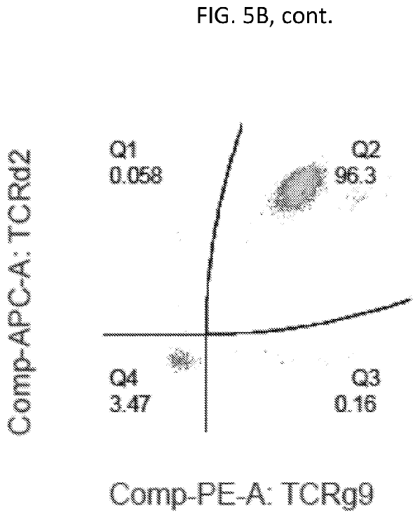

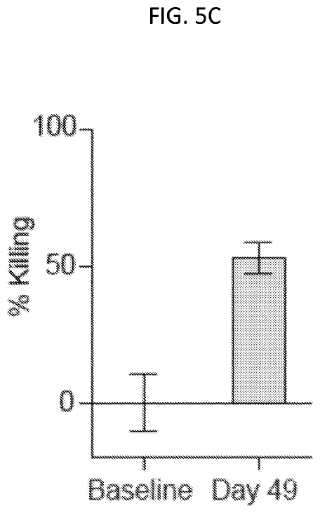

Patient dosing, sample collection, and flow cytometry: Patient 02 received 3×10⁷ CFU of live-attenuated Listeria monocytogenes (strain LmΔactAΔinlB.EGFRvIII.NYESO1) intravenously on study days 0, 28, and 49. Sodium heparin anticoagulated blood was stained for flow cytometry, and the frequency of the indicated T cell subsets was determined. Absolute lymphocyte frequency was obtained by CBC performed in the clinical hematology lab and used to calculate the absolute number of Vy9+Vδ2+ T cells per mL of blood (FIG. 5A). Blood from study day 56 showing the frequency of Vy9+Vδ2+ T cells within the total lymphocyte and CD3+ T cell populations (FIG. 5B).

Cytotoxicity assay: SK-BR3 target cells were stained for 20 min with IncuCyte CytoLight Rapid Green, plated in DMEM media, and incubated with or without 1 μg/ml of a bispecific anti-erbB2×anti-CD3 (anti-erbB-2 (Her-2/neu) [4D5-8 (trastuzumab)], Mouse IgG2a hCD3ε KIH antibody) for 2 hours at 37 °C, 5% CO₂. SK-BR3 is a human breast cancer cell line that overexpresses Her2. After 2 hours of incubation, PBMCs obtained from Patient 02 were added at a ratio of 5:1 (PBMC: Target cells). Pre-treatment and day 56 samples were used to evaluate cytotoxicity. Images were taken hourly in a CellCyte X Cell Analyzer, and confluence values were recorded. To determine the % killing, the confluence values obtained after 8 hours in the presence of the bispecific antibody were compared against those obtained without the antibody for each sample.

The patient PBMC following Listeria monocytogenes treatment (day 49) were more able to kill cancer cells via a bispecific antibody than that same patient's PBMC before treatment (FIG. 5C). The response is normalized to cells without the bispecific antibody, indicating that the antibody guided the cytotoxicity.

### Example 3

### Treatment of Cancer

This example describes methods of treating a subject with B cell lymphoma using a microbial conditioning regimen and a bispecific T cell engager. One of skill in the art will recognize that methods that deviate from these specific methods can also be used to successfully treat a subject with lymphoma, or another cancer.

Patients (such as patients having B cell lymphoma) are treated with a conditioning regimen of live attenuated Listeria monocytogenes, followed by treatment with Mosunetuzumab, a humanized anti-CD20/anti-CD3 bispecific antibody targeting CD20-expressing cells in patients with CD20+ B cell lymphoma. Patients are screened for expression of CD20 within the malignant lymphoma cell population. Eligible patients are treated i.v. on Day 0 with 3×10⁷ CFU of the live attenuated Listeria monocytogenes. Peripheral blood is collected on Day 21 and the frequency of Vγ9Vδ2+ T cells is determined. Patients with >5,000 Vγ9Vδ2+ cells/mL are eligible to begin treatment with Mosunetuzumab. Patients with <5,000 Vγ9Vδ2+ cells/mL receive additional treatment(s) with the live attenuated Listeria monocytogenes in 21-day intervals until the frequency of Vγ9Vδ2+ cells is >5,000 cells/mL. Twenty-two days after the last treatment with the live attenuated Listeria monocytogenes, patients are pretreated with 20 mg dexamethasone, 50 mg of diphenhydramine hydrochloride, and 1,000 mg of paracetamol. Patients then begin Cycle 1 and receive 1 mg of Mosunetuzumab. Seven days later, patients receive 2 mg of Mosunetuzumab. Seven days later, patients receive 60 mg of Mosunetuzumab. Cycles 2-8 involve infusion of 60 mg of Mosunetuzumab every 21 days thereafter up to 8 cycles total. Prior to each infusion of Mosunetuzumab, peripheral blood is collected and the frequency of Vγ9Vδ2+ cells is determined. If the frequency of Vγ9Vδ2+ cells is >1,000 cells/mL, the next dose of Mosunetuzumab is administered. If the frequency of Vγ9Vδ2+ cells is <1,000 cells/mL, a rest period of 21 days from the last Mosunetuzumab treatment is observed, after which the patient is re-treated with the 3×10⁷ CFU of the live-attenuated Listeria monocytogenes. Twenty-one days after bacteria treatment, bispecific treatment is resumed on a weekly schedule as above.

Patients are monitored during and following treatment for ECOG performance status, weight, vital signs, routine hematologic, chemical and endocrine parameters, immune cell subsets in the peripheral blood, tumor cell burden, and survival. A decrease in tumor burden or an increase in survival are indicators of efficacy of the treatment.

### Example 4

### Treatment of Infectious Disease

This example describes methods of treating a subject infected with Human Immunodeficiency Virus (HIV) using a microbial conditioning regimen and a bispecific T cell engager. One of skill in the art will recognize that methods that deviate from these specific methods can also be used to successfully treat a subject with HIV, or another infectious disease.

Patients (such as patients having HIV) are treated with a conditioning regimen of live attenuated Listeria monocytogenes, followed by treatment with a TCR/anti-CD3 bispecific fusion protein targeting HIV-infected cells via recognition of SL9 (from gag p17) HLA-A*02:01 peptide complexes (referred to hereafter as the bispecific). Eligible patients are treated i.v. on Day 0 with 3×10⁷ CFU of the live attenuated Listeria monocytogenes. Peripheral blood is collected on Day 21 and the frequency of Vγ9Vδ2+ T cells is determined. Patients with >5,000 Vγ9Vδ2+ cells/mL are eligible to begin treatment with the bi-specific. Patients with <5,000 Vγ9Vδ2+ cells/mL receive additional treatment(s) with the live attenuated Listeria monocytogenes in 21 day intervals until the frequency of Vγ9Vδ2+ cells is >5,000 cells/mL. Twenty two days after the last treatment with the bacteria, patients receive 20 μg of the bispecific. Seven days later, patients receive 30 μg of the bispecific. Seven days later, patients receive 68 μg of the bispecific. Patients are monitored for at least 16 hours after each dose of the bispecific. Prior to each infusion of the bispecific, peripheral blood is collected and the frequency of Vγ9Vδ2+ cells is determined. If the frequency of Vγ9Vδ2+ cells is >1,000 cells/mL, the next dose of the bispecific is administered. If the frequency of Vγ9Vδ2+ cells is <1,000 cells/mL, a rest period of 14 days from the last bispecific treatment is observed, after which the patient is re-treated with the 3×10⁷ CFU of the bacteria. Twenty-one days after bacteria treatment, bispecific treatment is resumed on a weekly schedule.

Patients are monitored during and following treatment for weight, vital signs, routine hematologic, chemical and endocrine parameters, immune cell subsets in the peripheral blood, viral load, and survival. A decrease in viral load indicates efficacy of the treatment.

In view of the many possible implementations to which the principles of the disclosure may be applied, it should be recognized that the illustrated examples should not be taken as limiting the scope of the invention. Rather, the scope of the invention is defined by the following claims. We therefore claim as our invention all that comes within the scope and spirit of these claims.

1. A method of treating a subject with disease or disorder, comprising:
administering to the subject a microbial conditioning regimen that activates, expands, and/or maintains a population of Vγ9Vδ2 T cells; and
administering to the subject a multi-specific T cell engager composition targeting an antigen expressed in the disease or disorder and one or more stimulatory receptors expressed by the Vγ9Vδ2 T cells,
thereby treating the disease or disorder.

2. The method of claim 1, wherein the microbial conditioning regimen is administered to the subject prior to administering the multi-specific T cell engager composition.

3. The method of claim 2, wherein the microbial conditioning regimen is administered to the subject about 7 days prior to administering the multi-specific T cell engager composition.

4. The method of claim 1, wherein the multi-specific T cell engager composition comprises a bi-specific T cell engager or a tri-specific T cell engager.

5. The method of claim 4, wherein the multi-specific T cell engager comprises:
a first portion comprising a binding domain that specifically binds to CD3; and
a second portion comprising a binding domain that specifically binds to an antigen expressed in the disease or disorder.

6. The method of claim 5, wherein the first portion of the multi-specific T cell engager comprises an anti-CD3 single chain variable fragment (scFv).

7. The method of claim 1, wherein the multi-specific T cell engager comprises:
a first portion comprising a binding domain that specifically binds to a TCRγ chain, a TCRδ chain, or both; and
a second portion comprising a binding domain that specifically binds to an antigen expressed in the disease or disorder.

8. The method of claim 4, wherein the second portion of the multi-specific T cell engager comprises a single chain variable fragment (scFv) that specifically binds to the antigen expressed in the disease or disorder or comprises a portion of a TCR targeting an antigen that is expressed in the disease or disorder.

9. A method of treating a subject with a disease or disorder, comprising:
administering to the subject a population of ex vivo expanded Vγ9Vδ2 T cells or a population of engineered Vγ9Vδ2 T cells expressing a chimeric antigen receptor (CAR) targeting the disease or disorder; and
administering to the subject a microbial conditioning regimen that activates, expands, and/or maintains the population of ex vivo expanded Vγ9Vδ2 T cells or the population of engineered Vγ9Vδ2 T cells.

10. The method of claim 9, wherein the microbial conditioning regimen is administered to the subject concurrent with or after administering the population of Vγ9Vδ2 T cells expressing the CAR targeting the disease or disorder.

11. The method of claim 1, wherein the microbial conditioning regimen comprises administering to the subject one or more doses of a composition comprising a bacterium that produces (E)-4-hydroxy-3-methyl-but-2-enyl pyrophosphate (HMBPP).

12. The method of claim 11, wherein the bacterium that produces HMBPP is a live attenuated bacterium.

13. The method of claim 11, wherein the bacterium that produces HMBPP is Listeria monocytogenes.

14. The method of claim 11, wherein the composition comprising the bacterium that produces HMBPP comprises about 1 to about 1×10¹³ colony forming units (CFU) of the bacteria.

15. The method of claim 1, wherein the microbial conditioning regimen is administered orally, intravenously, subcutaneously, intradermally, or intranasally.

16. The method of claim 1, wherein the disease or disorder is cancer or an infectious disease.

17. The method of claim 16, wherein the cancer is a solid tumor or a hematological malignancy.

18. The method of claim 17, wherein the cancer is a solid tumor comprising lung cancer, prostate cancer, breast cancer, ovarian cancer, gastric cancer, pancreatic cancer, or melanoma.

19. The method of claim 17, wherein the cancer is a hematological malignancy comprising B cell lymphoma, multiple myeloma, acute lymphoblastic leukemia, acute myeloid leukemia, or non-Hodgkin's lymphoma.

20. The method of claim 16, wherein the subject has cancer and further comprising administering to the subject one or more therapies selected from the group consisting of surgery, radiation treatment, chemotherapy, and immunotherapy.
